(19) United States
(12) Patent Application Publication
(10) Pub. No.: US 2007/0057083 A1
(43) Pub. Date: Mar. 15, 2007

(54) Method for producing an aerosol and injector unit

(76) Inventor: Detlef Bolz, Bad Buchau (DE)

Correspondence Address: FLYNN THIEL BOUTELL & TANIS, P.C., 2026 RAMBLING ROAD, KALAMAZOO, MI, 49008-1631, US

(21) Appl. No.: 10/575,799

(22) PCT Filed: Oct. 21, 2004
(86) PCT No.: PCT/EP04/11898

§ 371 Date: Apr. 13, 2006

(30) Foreign Application Priority Data
Oct. 21, 2003 (DE) 10349642.4

(51) Int. Cl. A24F 25/00; B05B 7/04; F23D 11/16; A61L 9/04

(52) U.S. Cl. 239/34; 239/398; 239/419.5

(57) ABSTRACT

A device for producing an aerosol, comprising a liquid line for a liquid flow and a transport gas line for a transport gas flow, comprising at least one injector unit wherein the liquid flow and transport gas flow can be mixed to form an aerosol, also comprising an aerosol line leading to an aerosol discharge arranged in the region of a tool, is known per se. According to the invention, the injector unit comprises flow guiding means for the transport gas flow which define a pressure loss for the transport gas flow, which is lower than the minimum possible pressure loss at the aerosol discharge, by increasing the flow speed and reducing the volume flow. The invention can be used to supply lubricating agents to machine tools.

The invention relates to a device for producing an aerosol, with a liquid line for a liquid flow and a transport, gas line for a transport gas flow, with at least one injector unit, in which the liquid flow and the transport gas flow can be mixed to form an aerosol, and with an aerosol line, which leads to an aerosol discharge arranged in the region of a tool.

The invention also relates to an injector unit for a device for producing an aerosol with at least one channel portion for a transport gas flow and with at least one channel region for the liquid flow.

Such a device for producing an aerosol and such an injector unit are also known from DE 101 04 012 C2. Aerosols serve in particular as lubricants and coolants for the machining of workpieces. To ensure reliable production of an aerosol for a relatively wide range of machine tools, the device for producing an aerosol has a throttle system, by means of which a transport gas, preferably compressed air, and a liquid, preferably oil, can be fed to the injector unit in controlled amounts. The compressed air and the oil are fed to the injector unit and vortexed in the injector unit, whereby the aerosol is produced. The aerosol produced in this way is conveyed into the aerosol line, where it emerges in the region of an aerosol discharge in the area of the tool at a corresponding machining location.

DE 101 39 950 A1 discloses a further device for producing aerosols in which a pressure gage is connected to the transport gas line, in this case to the compressed air line. Depending on the values sensed by the pressure gage, a differential pressure sensing device is adapted.

The object of the invention is to provide a device for producing an aerosol and an injector unit of the type mentioned at the beginning by means of which continuous production of an aerosol can be achieved independently of the machine tool used.

For the device for producing an aerosol, this object is achieved by the injector unit having flow conducting means for the transport gas flow which define a sucking-in and atomizing function for the liquid flow when there is a pressure loss for the transport gas flow which is less than a minimum possible pressure loss at the aerosol discharge. The fact that there is only an extremely small pressure differential in the region of the injector unit between the inlet side and the outlet side means that continuous feeding of transport gas and, accordingly continuous aerosol production, can be achieved, irrespective of whether the aerosol discharge has a great pressure loss or only an extremely small pressure loss in the area of the tool itself. The invention allows the aerosol production to be at least largely independent of the transport gas flow. Even if little transport gas is used up, the continuous aerosol production is maintained. The continuous aerosol production has the effect that there are virtually no pressure fluctuations. The certainty of the process is increased, in particular for a continuous lubricant feed for machining. The aerosol produced serves in particular for lubricating and cooling machine tools at a corresponding machining location of a workpiece.

In a refinement of the invention, a channel portion for the transport gas flow and a channel region for the liquid flow are arranged coaxially in relation to each other within the injector unit. The channel portion and the channel region preferably have directions of flow that are parallel to each other.

In a further refinement of the invention, the channel portion for the transport gas flow is configured as an annular channel concentrically surrounding the channel region of the liquid flow, and the flow conducting means comprise an annular constriction at the level of a stub-like end region of the channel region of the liquid flow, which together with an outer casing of the end region defines an annular gap. The liquid flow is preferably fed centrally, so that the transport gas, in particular compressed air, is directed past the outside of the channel region for the liquid flow. The end region of the chanel region of the liquid flow forms a breakaway edge. Since the constriction of the annular channel for the transport gas flow is provided at the level of the end region, an extremely small annular gap is produced here between the end region and the constriction. This small annular gap leads to a high speed of the transport gas at the atomizing location, that is at the breakaway edge, with at the same time an extremely low volumetric flow of transport gas. A great sucking and atomizing capacity is obtained with an extremely small differential pressure, with respect to a pressure differential between an inlet side, seen in the direction of flow, of the injector unit and an outlet side, which leads to the aerosol line.

In a further refinement of the invention, the annular gap is configured with dimensioning of <0.5 mm, preferably of approximately 0.1 mm. This makes it possible to achieve a smaller or at least equal pressure loss in the region of the aerosol discharge in the area of the injector unit, even when there are extremely small channel cross sections in the region of the aerosol discharge, and as a result ensure continuous aerosol production irrespective of the machine tool used.

In a further refinement of the invention, pressure sensing means are provided in the region of the transport gas line and in the region of the aerosol line, and a control unit is provided, which, depending on a comparison of actual pressure values sensed by the pressure sensing means with set pressure differential values stored in a set-value memory on the basis of various parameters for different machining operations, controls a differential pressure between the pressure in the transport gas line and the pressure in the aerosol line. As a result, it is possible to achieve a differential pressure control, whereby the amount of aerosol produced can be changed. In this case, the pressure differential between a pressure on the inlet side within the transport gas line and a pressure on the outlet side within the aerosol line is controlled.

In a further refinement of the invention, the control unit is assigned a control program, which activates at least one functional unit of the device with different control commands and in each case performs differential pressure measurements, and subsequently a comparison of the sensed actual values of the differential pressure measurements with corresponding set values of the set-value memory is performed, and finally a preselection of appropriate parameters is made from the set-value memory. This preselection may be displayed to the operator in a corresponding display unit. The display unit is preferably combined with a data input unit, so that the operator can make the desired individual parameter combination from the preselection indicated. Starting the control program has the effect in particular that the aerosol producer, as a functional unit of the device, is started and that the differential pressures at the corresponding locations are measured. By comparison with the values stored in the set-value memory, a preselection can be made from the entire block of parameter combinations stored in the set-value memory. This preselection is displayed to the operator, minimum and maximum parameter combinations being identified, so that the operator can select whether little or considerable lubricating medium is to be conveyed, or whether a mid-value between these minimum and, maximum values is to be selected. The control unit is preferably assigned a data input unit, by means of which parameters of a tool to be used can be input by an, operator, and a data processing program is provided as a control program, which, depending on input parameters of the data input unit, performs a comparison with all the parameters stored in the set-value memory and prescribes a preselection of appropriate parameter combinations made to match the tool to be used, from which the operator selects a desired parameter combination. As a result, depending on the tool to be used, an operator is given assistance, in that a preselection of appropriate parameter combinations is made, from which the operator then selects the desired parameters. The data input unit is preferably assigned a display unit, in which the preselection of the data processing program is made evident to the operator. The operator is advantageously displayed a minimum set of parameters and a maximum set of parameters, i.e. a corresponding parameter combination. The minimum set of parameters preferably means the setting with which least lubricating medium is conveyed. The maximum set of parameters corresponds to the setting with which most lubricating medium is conveyed. The fact that the overall system of the device, that is to say from the aerosol producer via the aerosol lines, the rotary leadthrough of the spindle, the tool holder and up to the cooling channel in the tool is considered, means that it is ensured that no mistaken inputs of inappropriate sets of parameters can be made. For each tool it is consequently ensured that, by the preselection of suitable parameter combinations, appropriate workpiece machining takes place in any event, achieving a desired machining result. The operator can assign from the preselected parameter combinations an individual set of parameters to the tool respectively used and, if appropriate, store it in the set-value memory or in the control unit of the device by corresponding storage. It is particularly advantageous if the preselection of corresponding parameter combinations is made by the operator fitting the tool to be used and starting an automatic run-in program, with which different activations are performed by means of suitable measuring instruments and storage methods, and the differential pressures occurring in each case for the different activations are measured. A corresponding comparison with stored values in the set-value memory allows appropriate differential pressures to be preselected, which are then assigned suitable further parameters by means of the data of the set-value memory. After completion of the run-in program, the operator can select a desired parameter combination from the appropriate parameter combinations displayed and so bring about desired open-loop or closed-loop control of the, device for a corresponding machining operation.

In a further refinement of the invention, a number of injector units are provided in parallel connection, to which a control branch of the transport gas line that can be controlled by an actuating element is respectively assigned, and the actuating elements can be activated by the control unit in such a way that at least one injector unit is permanently functioning. As a result, the liquid content within the aerosol can be controlled. Compressed air is preferably provided as the transport gas and oil is preferably provided as the liquid. Consequently, the desired amount of oil within the aerosol can be controlled.

In a further refinement of the invention, the activation of the actuating elements by the control unit takes place in dependence on corresponding control defaults of the set-value memory. The set-value memory preferably has different liquid contents within the aerosol for specific, typical machining operations and for different tools. Corresponding parameters for different tools and different machining operations are prescribed in the set-value memory and can be adapted by activation of the actuating elements and corresponding switching on or off of at least one injector unit. There is preferably a dependence of this activation on the differential pressure control.

For the injector unit, the object on which the invention is based is achieved by the channel portion for the transport gas flow being configured as an annular channel concentrically surrounding the channel region of, the liquid flow, and by the flow conducting means comprising an annular constriction at the level of a stub-like end region of the channel region of the liquid flow, which together with an outer casing of the end region defines an annular gap. This creates the previously already mentioned throttling effect with an extremely small pressure loss. The transport gas flow and the liquid flow are preferably directed in the same direction.

In a refinement of the injector unit, the channel portion for the transport gas flow narrows in a funnel-shaped manner in the direction of flow toward the constriction, and an aerosol chamber portion lying downstream of the end region widens in a correspondingly funnel-shaped manner in the direction of flow. The constriction is consequently connected symmetrically upstream and downstream to corresponding flow channel regions. The funnel shape is preferably formed in each case by a conical shaping. The constriction is preferably represented by a cylinder wall. The stub-shaped end region for the liquid flow is preferably realized by a hollow-cylindrical pipe stub. A breakaway edge of the end region is configured in a preferred way in a peripherally annular and sharp-edged manner.

Further advantages and features of the invention emerge from the claims and from the description which follows of a preferred exemplary embodiment of the invention, which is represented on the basis of the drawings.

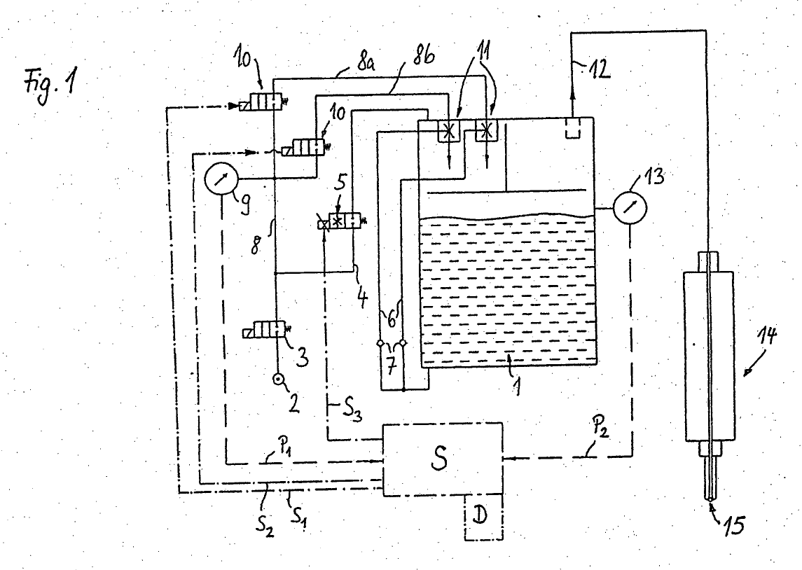

FIG. 1 schematically shows an embodiment of a device according to the invention for producing an aerosol in a block diagram and

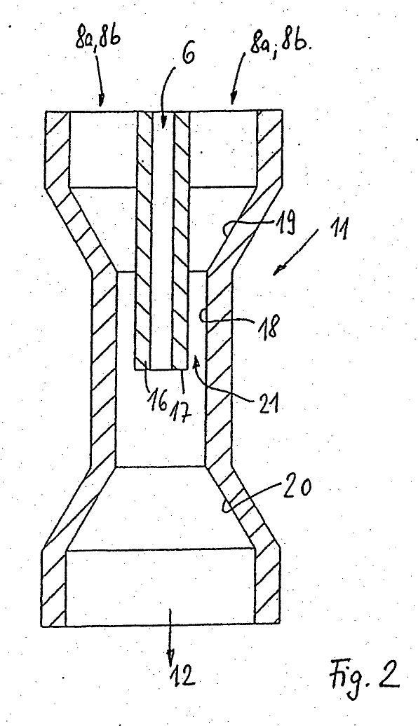

FIG. 2 shows an injector unit for the aerosol producing device as shown in FIG. 1 in an enlarged, schematic sectional representation.

A device for producing an aerosol as shown in FIG. 1 has a liquid container, in the present case in the form of an oil container. The liquid container 1 is only partially filled with liquid. An upper region of the liquid container 1 serves as an aerosol chamber, as described in more detail below. The device is also provided with a transport gas source 2, the transport gas taking the form of compressed air in the present exemplary embodiment. The compressed air source 2 is adjoined by a compressed air line 2, which serves as a transport gas line and is provided with a main valve 3 directly downstream of the compressed air source 2, in the direction of flow. The main valve 3 can be transferred into a blocking position or into a through-flow position.

The device for producing an aerosol is provided with a number of injector units 11, in the case of the present exemplary embodiment with two injector units 11. The injector units 11 are arranged within the aerosol-forming portion of the liquid container 1, referred to as the aerosol chamber. Branching away from the aerosol-chamber is an aerosol line 12, which leads to a machine tool 14. The aerosol line 12 opens out in the region of a machining location of the tool at at least one outlet opening of the tool, which defines an aerosol discharge 15.

The two injector units 11 serve the purpose of producing the aerosol from a fed compressed air flow and a sucked-in oil flow, in that the oil flow is atomized by the compressed air. For feeding the compressed air, the compressed air line 8 is subdivided into two line branches 8a, 8b, which are respectively led to one of the two injector units 11. Each injector unit 11 is also adjoined by an oil line 6. The compressed air flow has the effect of producing within each injector unit 11 a negative pressure, which sucks the oil in from the respective oil line 6. The oil line 6 is also connected to the liquid container 1 in a lower region of the liquid container 1 by means of a removal stub and subsequently branches into two line portions, which respectively lead to one of the two injector units 11. Provided in each line portion of the oil line 6 is a nonreturn valve 7, which prevents the return of oil into the liquid container 1 and moreover provides defined pressure conditions in each line branch of the oil line 6.

Each line branch 8a, 8b of the compressed air line is respectively assigned an adjusting valve 10, referred to as a nozzle valve, which releases or blocks the respective line branch 8a, 8b for a through-flow.

Fitted in the compressed air line 8 at the level of the branch of the two line branches 8a, 8b from the compressed air line 8, and consequently downstream of the actuating valves 10, is a pressure transducer 9, which can sense pressure values of the compressed air line 8. The aerosol chamber of the liquid container 1 is also assigned a pressure transducer 13, which picks up pressure values within the aerosol chamber which coincide with the respective pressure within the aerosol line 12. Consequently, the pressure upstream of the injector units 11 and downstream of the injector units 11, in each case seen in the direction of flow, is sensed by the pressure transducers 9, 13.

The two line branches 8a, 8b of the compressed air line are connected parallel to each other. In the same way, the two line branches of the oil line 6 are also led parallel to each other.

Led between the compressed air line 8 and the aerosol chamber is a pressure-equalizing line 4, which is assigned a control valve 5, in the present case in the form of a proportional valve. By means of this control valve 5 it is possible to control pressure differentials between the pressure in the compressed air line 8 and the pressure in the aerosol chamber.

For activating the control valve 5 and for activating the actuating valves 10, a central control unit S is provided, connected to which are corresponding signal lines P₁, P₂ of the two pressure transducers 9, 13. The central control unit S is provided with a data memory D, which prescribes set values in the form of various parameters for different machining operations and different tools. Corresponding sets of parameters prescribe differential pressure values suitable for different types of tool, and consequently for different channel cross sections of the tools in the region of the aerosol discharge 15 and for different types of machining, such as drilling, milling and the like, or for different workpiece materials, with respect to the difference between the pressure in the compressed air line 8 and the pressure in the aerosol line 12. The respectively available parameters for the corresponding machining operation are input by an operator into a data input unit (not represented any more precisely) of the control unit S. Alternatively, it is also possible that, by corresponding sensor means, the device can itself sense which parameters are used in the case of the respective machining operation. This is not discussed in any more detail at this point.

In the case of the exemplary embodiment represented, the corresponding parameter default is set by manual operation. The prescribed parameters are compared in the central control unit S with the sets of parameters available in the data memory D. Depending on the result of the comparison, on the one hand the actuating valves 10 for the switching on or off of the line branches 8a, 8b of the compressed air line are activated via control lines S₁, S₂. On the other hand, the control valve 5, configured as a proportional valve, is activated, in order to control the corresponding pressure differential control between the compressed air line 8 and the aerosol line 12 or the aerosol chamber within the liquid container 1. The fact that actual values of the respectively available pressures in the compressed air line 8 and in the aerosol line 12 are sensed by means of the pressure transducers 9, 13 and fed to the control unit S via the signal lines P₁, P₂ allows the control unit S to carry out a constant comparison with the differential pressure values that are stored in the data memory D and prescribed for specific sets of parameters, and, depending on the result of the comparison, activate the control valve 5.

In the case of the exemplary embodiment represented, as shown in FIG. 1, only two injector units 11 are provided for reasons of overall clarity. In the case of practical exemplary embodiments, however, more than two injector units 11 are also connected in parallel, depending on which amount of oil is to be mixed in with the compressed air.

In a way not represented any more precisely, the liquid container 1 is connected to a liquid reservoir and provided with a filling level monitor, in order to make it possible for the oil to be topped up in good time, in particular in the liquid container 1 serving as the aerosol container.

Each injector unit 11 is configured in a way according to the representation as shown in FIG. 2. As can be seen from FIG. 2, a central oil feed is provided. The compressed air flow is led past the oil feed on the outside. For this purpose, the injector unit has a channel region which is defined by a hollow-cylindrical pipe portion and into which the respective line branch of the oil line 6 opens. The central channel region 16 is concentrically enclosed by an annular channel portion into which the respective line branch 8a or 8b of the compressed air line opens. The annular channel for the compressed air flow has a conical narrowing portion 19, which leads to a constriction 18. The constriction 18 is formed by a cylindrical wall portion, which coaxially encloses a cylindrical outer casing of the channel region for the oil flow. Between the constriction 18 and the cylindrical outer casing of the channel region for the oil flow there remains an annular gap 21, which is made to be extremely small and in the case of the exemplary embodiment represented is about 0.1 mm wide.

At the axial level of the constriction 18, the channel region for the oil line 6 ends and forms an end region 16. The end region 16 is provided at its outer rim with a peripheral and sharp-edged breakaway edge 17.

At a distance beneath the breakaway edge 17, and consequently downstream, there is an aerosol chamber portion which opens into the aerosol chamber of the liquid container 1 and consequently into the aerosol line 12. The aerosol chamber portion has a conical widening region 20, which is dimensioned in a way corresponding to the conical narrowing region 19 of the injector unit 11.

The extremely narrow annular gap 21 creates in the region of the breakaway edge 17 an extremely high flow speed of the compressed air with at the same time an extremely low volumetric flow. The negative pressure created at the end region 16 of the oil line 6 leads to the oil droplets being sucked in and atomized. The high flow speed and the simultaneously low volumetric flow have the effect that only a very small differential pressure is obtained in the region of the injector unit 11, i.e. at the atomizing location, i.e. an extremely small pressure loss. The injector unit 11, which is also referred to as atomizer nozzles, are made so small that the pressure loss in the region of an injector unit 11 is in any event smaller than the pressure loss in the region of the aerosol discharge 15, even if a machine tool with an extremely small channel cross section in the outlet region of the lubricant or coolant, i.e. in the region of the aerosol discharge 15, is provided. This ensures that permanent and continuous aerosol production is possible. The actuating valves 10 of the line branches 8a, 8b of the compressed air line 8 are activated in such a way that at least one of the actuating valves 10 is always open, so that a permanent compressed air flow through at least one injector unit 11 is ensured.

This would not be possible if—as customary in the prior art—the pressure loss in the region of an injector unit were greater than a minimum possible pressure loss in the region of the aerosol discharge.

Depending on the proposed sets of parameters of the data memory D and the manually input parameters respectively available at the machine tool 14, the control unit S distinguishes between smallest, small, moderate and large cooling or lubricating channels and prescribes different differential pressures for these different cooling or lubricating channels. Moreover, it describes how many injector units 11 are to be switched on, i.e. how large the oil content within the aerosol is to be.

1. A device for producing an aerosol, with a liquid line for a liquid flow and a transport gas line for a transport gas flow, with at least one injector unit, in which the liquid flow and the transport gas flow can be mixed to form an aerosol, and with an aerosol line, which leads to an aerosol discharge arranged in the region of a tool, characterized in that the injector unit (11) has flow conducting means (16 to 18) for the transport gas flow which define a sucking-in and atomizing function for the liquid flow when there is a pressure loss for the transport gas flow which is less than a minimum possible pressure loss at the aerosol discharge (15).

2. The device as claimed in claim 1, characterized in that a channel portion for the transport gas flow and a channel region for the liquid flow are arranged coaxially in relation to each other within the injector unit (11).

3. The device as claimed in claim 2, characterized in that the channel portion for the transport gas flow is configured as an annular channel concentrically surrounding the channel region of the liquid flow, and in that the flow conducting means comprise an annular constriction (18) at the level of a stub-like end region (16) of the channel region of the liquid flow, which together with an outer casing of the end region (16) defines an annular gap (21).

4. The device as claimed in claim 3, characterized in that the annular gap (21) is configured with dimensioning of <0.5 mm, preferably of approximately 0.1 mm.

5. The device as claimed in claim 1, characterized in that pressure sensing means (9, 13) are provided in the region of the transport gas line (8) and in the region of the aerosol line (12), and in that a control unit S is provided, which, depending on a comparison of actual pressure values sensed by the pressure sensing means (9, 13) with set pressure differential values stored in a set-value memory (D) on the basis of various parameters for different machining operations, controls a differential pressure between the pressure in the transport gas line (8) and the pressure in the aerosol line (12).

6. The device as claimed in claim 5, characterized in that the control unit is assigned a control program, which activates at least one functional unit of the device, in particular an aerosol producer, with different control commands and in each case performs differential pressure measurements by means of the pressure sensing means, and in that a comparison of the sensed actual values of the differential pressure measurements with corresponding set values of the set-value memory is performed, and finally a preselection of appropriate parameters is made from the set-value memory.

7. The device as claimed in claim 5, characterized in that a number of injector units (11) are provided in parallel connection, to which a control branch (8a, 8b) of the transport gas line (8) that can be controlled by an actuating element (10) is respectively assigned, and in that the actuating elements (10) can be activated by the control unit (S) in such a way that at least one injector unit (11) is permanently functioning.

8. The device as claimed in claim 7, characterized in that the activation of the actuating elements (10) by the control unit (S) takes place in dependence on corresponding control defaults of the set-value memory (D).

9. An injector unit for a device for producing an aerosol with at least one channel portion for a transport gas flow and at least one channel region for a liquid flow, characterized in that the channel portion for the transport gas flow is configured as an annular channel concentrically surrounding the channel region of the liquid flow, and in that the flow conducting means comprise an annular constriction (18) at the level of a stub-like end region of the channel region of the liquid flow, which together with an outer casing of the end region (16) defines an annular gap (21).

10. The injector unit as claimed in claim 9, characterized in that the channel portion for the transport gas flow narrows in a funnel-shaped manner in the direction of flow toward the constriction (18) and an aerosol chamber portion (20) lying downstream of the end region (16) widens in a correspondingly funnel-shaped manner in the direction of flow.
